(19) United States
(12) Patent Application Publication
(10) Pub. No.: US 2025/0032819 A1
(43) Pub. Date: Jan. 30, 2025

(54) DISTRIBUTED MULTI-CAMERA REAL-TIME TUMOR POSITIONING AND TRACKING METHOD BASED ON VISUAL POSITION-AWARE MARK AND TRACKING SYSTEM THEREOF

(71) Applicant: Fujian Medical University Union Hospital, Fuzhou City (CN)

(72) Inventors: Xiaobo LI, Fuzhou City (CN); Bingwei HE, Fuzhou City (CN); Fen ZHENG, Fuzhou City (CN); Zhiyu YANG, Fuzhou City (CN)

(21) Appl. No.: 18/649,168

(22) Filed: Apr. 29, 2024

(30) Foreign Application Priority Data
Jul. 28, 2023 (CN) 2023109367462

(51) Int. Cl. A61N 5/10; G06T 7/00; G06T 7/246; G06T 7/73

(52) U.S. Cl. CPC A61N 5/1049; G06T 7/0012; G06T 7/246; G06T 7/73; A61N 2005/1059; A61N 2005/1074; G06T 2207/30096; G06T 2207/30204

(57) ABSTRACT

A distributed multi-camera real-time tumor positioning and tracking method based on a visual position-aware mark and tracking system thereof includes the steps: carrying out the coding of a high-density self-identification visual mark and obtaining a specific Hella code; detecting and identifying the Hella code; based on the identified Hella code, carrying out spatial positioning and full-view registration on the marked mark features, and applying the Hella code to tumor positioning and tracking. According to the method, high-precision sensing of the position of the patient is realized; by analyzing the medical image data, the position, posture, and anatomical structure information of the patient can be accurately determined, and accurate positioning and navigation are provided for accurate radiotherapy.

### TECHNICAL FIELD

The present invention pertains to the domain of visual positioning technology and is specifically concerned with a method for real-time tumor positioning and tracking, which employs a distributed multi-camera system equipped with visual position-aware markers and a tracking system thereof.

### BACKGROUND

A location-aware marker is a technique enabling a system to precisely determine the position of an object or area by marking it directly. These markers are created using a Hella code generator, featuring multiple self-recognizing perceptual units. This ensures that the object or area can be localized and tracked as long as at least one of these units is captured by a camera.

Existing methods usually rely on closed patterns such as characters and QR codes in realizing self-recognition, and self-recognition is contradictory to high confidentiality. Therefore, one of the key scientific issues to be addressed is how to create novel coding methods to organize self-recognition patterns with the features themselves to satisfy the high density and self-recognition required for radiotherapy localization.

The redundancy of high-density self-recognition patterns alleviates the need for high recall rate for localization, but raises the need for precision rate, which requires a more stringent verification process. Therefore, how to design the detection and recognition process to harmonize the speed and precision rate to meet the localization demand during radiotherapy is one of the key scientific issues to be addressed.

To adapt to the more flexible motion of the localization target and reduce the viewpoint sensitivity, the mark features are sometimes distributed in multiple directions, resulting in the features not being able to be registered synchronously in a single viewpoint. Therefore, how to achieve full-view registration of mark features based on multi-view image stitching and optimization is one of the key scientific problems to be solved.

### SUMMARY

The present invention provides a distributed multi-camera real-time tumor positioning and tracking method based on visual position-aware mark and tracking system thereof to solve the above problem.

The present invention is realized by the following technical solutions:

A distributed multi-camera real-time positioning and tracking method based on visual position-aware mark, the localization and tracking method comprising:

Encoding a high-density self-recognizing visual mark to obtain a specific Hella code;
Detecting and recognizing the Hella code;
Spatial localization and full-view registration of mark features marked therewith based on the recognized Hella code;
Application of the Hella code to tumor positioning and tracking based on the above.

Further, the step of encoding the high density self-identification visual mark to obtain the specific Hella code comprises generating a self-identification unit and a splicing self-identification unit; the generating a self-identification unit specifically includes: using an intersection point as a basic feature, using every 3×3 feature of the self-identification pattern as a basic unit, and compiling an identification number by using the orientation of the intersection point; starting from an upper left corner feature by using a Boolean value of a central feature orientation as a first bit, filling subsequent bits in a clockwise direction to form a 9-bit feature value, and converting the 9-bit feature value into a decimal identification number; and rotating the self-identification pattern clockwise three times to respectively generate identification numbers;

This is achieved by shifting and inverting, viz:

$${\int }_{i}^{90} = \{ \begin{matrix}{f}_{i} & i = 0 \\ \sim {f}_{7 + i} & 3 > i > 0 \\ \sim {f}_{i - 2} & i > 3\end{matrix}$$

Wherein fᵢ denotes the i-th bit of the identification number f, f⁹⁰ denotes the identification number of the self-identification unit after clockwise rotation, and ˜ is an inverse operation.

Further, the splicing self-identification unit is specified as, exhaustively enumerating all 9-bit feature values and filling the unit pool; subsequently, traversing the unit pool, discriminating and removing rotationally duplicated and rotationally ambiguous units based on shift and inverse operations of the identification number; and finally, iteratively splicing the self-identification pattern based on the common regions of neighboring units by establishing a connection table.

Further, the detecting and recognizing the Hella code includes basic feature detection of the visual marks and decoding of the self-recognizing units; the basic feature detection of the visual marks specifically, comprises initial screening of the features, ridge localization, template validation, and circular validation, in a fast-to-slow, layer-by-layer manner, taking into account real-time and high accuracy,
wherein the feature initial screening is based on the inverse color characteristics of intersection points, checking 8 sampling points and calculating the contrast value to quickly exclude non-intersection point pixels:

$$\{ \begin{matrix}{G}_{outer} = {max}_{i} ( {I}_{i} - {I}_{i + 4} ) - {min}_{i} ( {I}_{i} - {I}_{i + 4} ) \\ {G}_{inner} = \vert {I}_{i} - {I}_{i + 4} \vert\end{matrix} i = 1 , … , 8$$

where Iᵢ is the luminance value of the i-th sampling point; locate the two ridges of the intersection feature based on the jump point location;
Correcting the sampling center to the center of the intersection with sub-pixel accuracy based on the intersection position of the ridges to generate the validation frame and extract the local pattern;
First, characterizing the intersection features in terms of the angle (θ1,θ2) of the ridges, looking up a table in the offline template generated based on multiple sampling, matching the table with the standard template, and validating the correlation; then, extracting the circular range connected by the intersection feature, calculating the sub-pixel luminance center of gravity, and validating the completeness of the circular circle; and, after all the validations are passed, outputting the image coordinates of the feature.

Further, the decoding of the self-recognition unit is specified as, firstly, organizing the detected feature points into a vertically and horizontally connected graph structure based on the ridge direction, assigning relative numbers (l, m, n);

Where I∈L is the number of the connected graph, and m and n are the temporary coordinates of the detected feature points in the graph structure l. Subsequently, all the 3×3 arrays are extracted, the identification numbers are calculated based on the intersection orientations, and the deviation of the temporary coordinates of the features from the absolute coordinates O₁,m,n is calculated by comparing with the key matrix;

Finally, a unique deviation O₁ is recognized for each graph structure based on the majority principle, viz:

O₁=Mode(0₁,m,n)

Wherein, Mode represents mode calculation; based on the deviation voting result, the detection features that Oₗ, m, n and Oₗ are not equal are excluded; thus, decoding of the self-identification unit is completed, and a unique identification number is assigned to each reliable detection feature.

Further, the spatial positioning and full-view registration of the mark features marked by the recognized sea-pull code includes spatial positioning of feature points, full-view registration of feature points, and spatial positioning of marks;

The spatial positioning of the feature points is specifically implemented by using at least four camera groups and based on the self-identification features detected in each view and camera group calibration data;

The full-view registration of feature points is specified as spatially localizing the feature points by taking full-view shots using camera encircling or target rotating, and searching for T and X that make the following formula optimal to complete the registration of the feature points:

Σ_{C}^{C}Σ_{P}^{P}λ_{c,p}(Y_{cp}−T_{c}X_{P})²
wherein cϵC is the observation viewpoint number and pϵP is the feature point number; T_{c} is the rotation translation matrix of viewpoint c, and X is the registration coordinates of the feature point group, both of which are optimization-seeking variables; Y_{c,p} is the spatial localization coordinates of the p-th feature point in the c-th viewpoint and λ_{c,p} is a Boolean value indicating whether or not the feature p has been observed in viewpoint c;

Finding the optimal X based on the 3D block leveling method and the optimal X is output as the result of feature point registration.

Further, the spatial localization of marks is specified as, the spatial localization of marks is a fusion of the feature point localization and the registration result, obtained by optimizing the following equation:

Σ_{P}^{P}Λ_{P}(X_{P}−DY_{P})²
wherein X is the registered coordinates of the feature point, Y is the spatial localization coordinates of the currently detected feature point; D is the mark positional attitude, an optimization-seeking variable; and the optimal positional attitude D is output as a spatial localization result of the mark.

Further, the applying the Hella code to real-time tumor positioning and tracking based on the above is specifically applied to real-time tumor positioning and tracking after control validation using a radiotherapy pose test;

The position-aware mark point and the metal markpoint (metallic mark point) are secured in the same mark point holder, and the position-aware mark point is affixed to the patient's skin at the portion of the thermoplastic membrane that has been removed. The metal mark point serves as an alignment reference during image guidance by the image guidance positioning system IGPS, and the position-aware mark point responds to the distance moved during the pendulum posing;

Image guidance with the IGPS corrects the patient's posing error, and the visual localization system tracks the patient's moving distance during this process, comparing the consistency of the patient's moving distance calculated by the two.

A distributed multi-camera real-time tumor positioning and tracking system based on visual position-aware mark, the localization and tracking system comprising:
An encoder for encoding a high-density self-recognizing visual mark to obtain a specific Hella code;
A recognition module for detecting and recognizing the Hella code;
A localization and registration module for spatial localization and full-view registration of its marked mark features based on the recognized Hella code.

Further, the positioning and tracking system is applied for tumor positioning and tracking.

The beneficial effects of the present invention are:

The present invention realizes high-precision perception of the patient's position; by analyzing medical image data, the patient's position, posture, and anatomical structure information can be accurately determined, providing accurate localization and navigation for precision radiation therapy.

The visual position-aware code of the present invention is capable of sensing changes in the position of the patient in real-time and making timely adjustments and corrections, and real-time image tracking and positional correction can be realized through integration with radiation therapy equipment.

The present invention innovatively adopts a position-aware mark as a visual reference object, which has a plurality of self-recognizing units of different sizes, and the camera can complete the tracking and localization thereof as long as it sees any one of the self-recognizing units; by designing an innovative pattern or logo for localization and tracking, it is more convenient and simpler to use, and it is easier to manipulate, recognize, and tracking.

The present invention proposes a distributed localization system, which can be arranged with a plurality of cameras in a treatment room, and the cameras can work with each other as well as individually, thus improving the reliability and accuracy of the localization system.

### BRIEF DESCRIPTION OF THE DRAWINGS

FIG. 1 illustrates a schematic diagram of a self-recognizing pattern of the present invention.

FIG. 2 illustrates a schematic diagram of rotational disambiguation and repetition of the present invention.

FIG. 3 illustrates a diagram of connectivity relationships and search order of the present invention.

FIG. 4 illustrates a flowchart of detection of basic features of the present invention.

FIG. 5 illustrates a schematic diagram of a full-view registration of the present invention

FIG. 6 illustrates a flowchart of a method of the present invention.

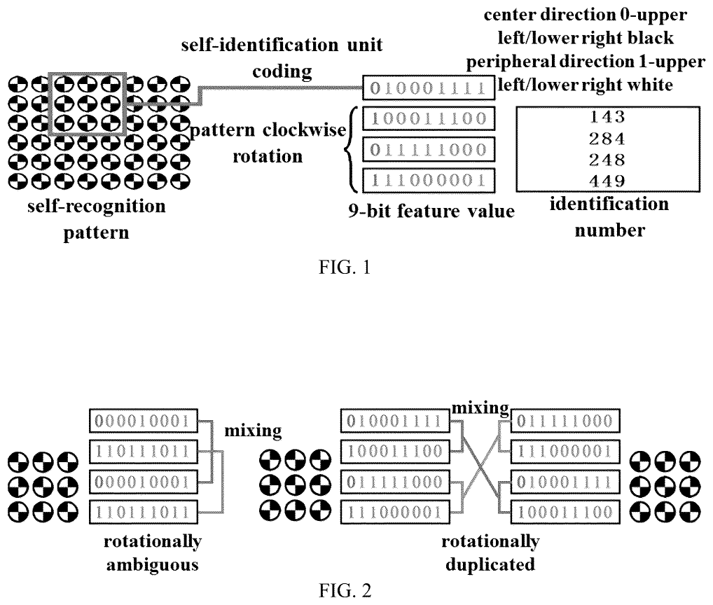

### DETAILED DESCRIPTION OF THE EMBODIMENTS

The following clearly and completely describes the technical solutions in the embodiments of the present invention concerning the accompanying drawings in the embodiments of the present invention, and obviously, the described embodiments are only a part of the embodiments of the present invention, but not all the embodiments. Based on the embodiments of the present disclosure, all other embodiments obtained by those skilled in the art without creative efforts fall within the protection scope of the present disclosure.

A distributed multi-camera real-time tumor positioning and tracking method based on visual location-aware mark, the localization and tracking method comprising:
Encoding a high-density self-recognizing visual mark to obtain a specific Hella code; encoding: comprising the generation of a self-recognizing unit, and splicing of a self-recognizing pattern;
Detecting and recognizing the Hella code; recognizing: including detection of basic features, decoding of the self-recognizing units;
Spatial localization and full-view registration of features of the mark marked therewith based on the recognized Hella code; localization and registration: including spatial localization of feature points, spatial localization of the mark, full-view registration of feature points, and calibration of the patient's body surface;
Applying the HELAC code to tumor positioning and tracking based on the above.

Further, the step of encoding the high-density self-identification visual mark to obtain the specific sea-pull code comprises: generating a self-identification unit and splicing the self-identification unit; the generating the self-identification unit specifically adopts an intersection point as a basic features, which is easy to detect and are significantly different from a natural feature, which is beneficial to improving the accuracy rate. In addition, false positives may be identified using circular envelope feature points while improving redundant representation of feature locations to improve positioning accuracy;

Each 3×3 feature of the self-identification pattern is a basic unit, and the identification number is compiled using the intersection point orientation; the Boolean value of the center feature orientation is the first one, the upper-left feature starts, and the subsequent bits are filled in the clockwise direction to form a 9-bit feature value, which is converted into a decimal identification number; To solve the problem of rotational ambiguity, the self-identification pattern is rotated clockwise three times, and the identification number is generated, respectively;

The specific generation method can be realized by shifting and inverting, viz:

$${\int }_{i}^{90} = \{ \begin{matrix}{f}_{i} & i = 0 \\ \sim {f}_{7 + i} & 3 > i > 0 \\ \sim {f}_{i - 2} & i > 3\end{matrix}$$

wherein fᵢ denotes the i-th bit (0 bit from the left) of the identification code f, f⁹⁰ denotes the identification code of the self-recognizing unit after clockwise rotation, and ˜ is an inverse operation.

Further, the splicing self-recognizing unit is specified as generating a self-recognizing pattern using splicing the self-recognizing unit, comprising a total of three steps of exhaustion, de-redundancy, and fusion; first, exhausting all 9-bit eigenvalues and filling in a unit pool; subsequently, traversing the unit pool to discriminate and remove rotationally duplicated, rotationally ambiguous units based on the identification number's shifting and inverse operations; and finally, based on the neighboring units of the common region, a connection table is established, and the self-recognizing pattern is iteratively spliced.

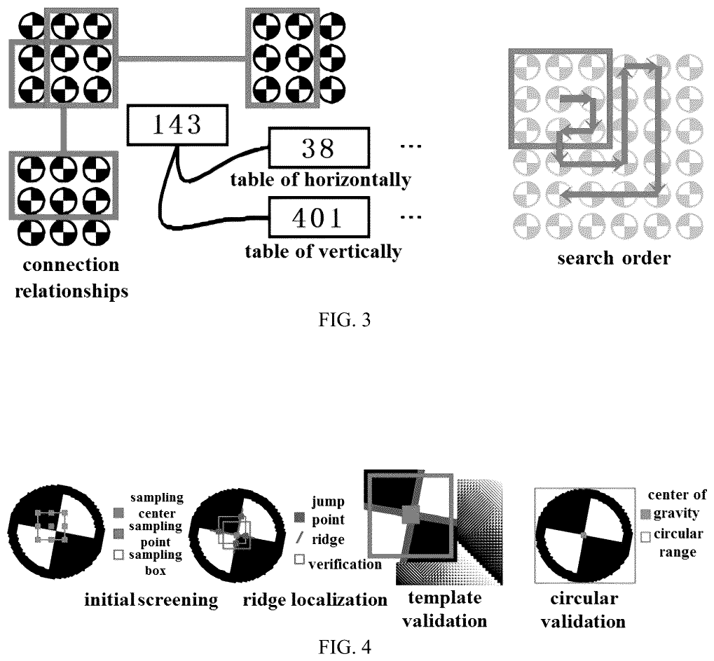

The meaning of rotational ambiguity and rotational duplication is shown in FIG. 2. The former will be removed from the pool of units and the latter will remain one. Horizontally or vertically connected self-recognizing units possess 3×2 or 2×3 common features as shown in FIG. 3. It is proposed to traverse the pool of units after redundancy and build a table of horizontally and vertically connected units based on identification numbers. The self-recognition pattern is spliced by an iterative search: the self-recognition pattern grows along the serpentine search route; each time, connectable units are taken out of the connection table and spliced to the current position; when there are no connectable units, a step back is taken to take out the untried connectable units from the connection table in the right place, and so on. The search process may be interrupted at any time to output the current largest rectangular self-recognizing pattern, accompanied by the output of a key matrix of 4 rotation angles, comprising the should-identifier.

Further, detecting and recognizing the Hella code comprises basic feature detection of the visual marks and decoding of the self-recognizing units; The basic feature detection of the visual marks is specifically the detection of the basic features of the visual marks comprises initial screening of the features, ridge localization, template verification, and circular verification, from fast to slow, layer by layer, taking into account real-time and high accuracy, as shown in FIG. 4.

Wherein, the feature initial screening is based on the inverse color characteristics of intersections, checking 8 sampling points and calculating the contrast value to quickly exclude non-intersection pixels:

$$\{ \begin{matrix}{G}_{outer} = {max}_{i} ( {I}_{i} - {I}_{i + 4} ) - {min}_{i} ( {I}_{i} - {I}_{i + 4} ) \\ {G}_{inner} = \vert {I}_{i} - {I}_{i + 4} \vert\end{matrix} i = 1 , … , 8$$

wherein Iᵢ is the luminance value of the i-th sampling point; if Gouter is sufficiently large and Ginner is sufficiently small, the sampling center is considered to have passed the initial screening; detecting luminance jump points in the sampling frame to exclude features with several jump points other than 4 or with an order of jumps that do not correspond to the expected order; and locating two ridges of the intersection feature based on the location of the jump points;

Correcting the sampling center to the center of the intersection with sub-pixel accuracy based on the intersection location of the ridges, generating a verification frame, and extracting the local pattern;

First, characterize the intersection feature in terms of the angle (θ1,θ2) of the ridges, look up the table in the offline template generated based on multiple sampling, match it with the standard template, and validate the correlation; then extract the circular range connected by the intersection feature, calculate the center of gravity of the sub-pixel luminance, and validate the completeness of the circular circle; and, after all the validations are passed, output the image coordinates of the feature.

Further, the decoding of the self-recognition unit is specified as that the decoding of the self-recognition unit is proposed to comprise three parts, namely, feature point organization, key retrieval, and coordinate bias voting; firstly, the detected feature points are organized based on the ridge direction into a vertically and horizontally connected graph structure, which is assigned a relative number (l, m, n);

Where IϵL is the number of the connected graph, and m and n are the temporary coordinates of the detected feature points in the graph structure l; subsequently, all the 3×3 arrays are extracted, the identification numbers are computed based on the intersection orientations, and the deviation of the temporary coordinates of the features from the absolute coordinates (the ranks they are placed in in the self-identification pattern) is computed Oₗ,m,n by comparing them with the key matrix;

Finally, a unique deviation O₁ is recognized for each graph structure based on the majority principle, viz:

O₁=Mode(O_{1,m,n})

Wherein, Mode denotes seeking the plurality; based on the result of the deviation voting, the detected features whose Oₗ,m,n are not equal to Oₗ are excluded; so far, the decoding of the self-recognition unit is completed, and a unique identification number is assigned to each reliably detected feature.

Further, the spatial localization and full-view registration of the mark features tagged therewith based on the recognized Hella code comprises spatial localization of the feature points, full-view registration of the feature points and spatial localization of the marks;

The spatial localization of the feature point is specified as the spatial localization of the feature point using at least a four-eye camera set, based on the self-identified features detected in each view with the camera set calibration data;

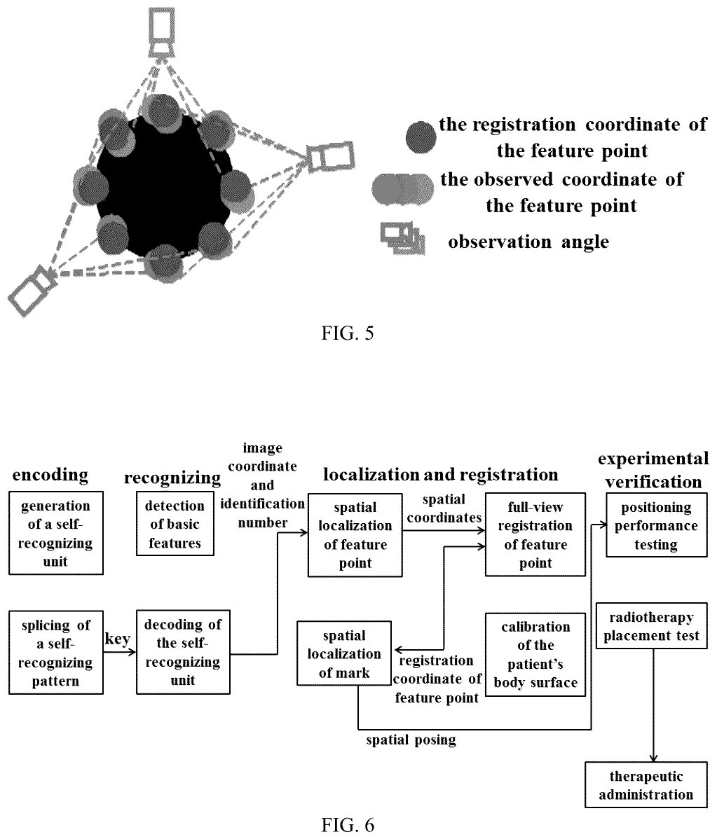

The full-view angle registration of feature points is specified as, as shown in FIG. 5, taking a cylindrical target as an example, spatially localizing the feature points by taking full-view angle shots using camera encircling or target rotating, and searching for T and X that make the following formula optimal to complete the registration of the feature points:

Σ_{C}^{C}Σ_{P}^{P}λ_{c,p}(Y_{c,p}−T_{c}X_{P})²
where cϵC is the observation viewpoint number and pϵP is the feature point number; Tc is the rotation translation matrix of viewpoint c and X is the registration coordinates of the feature point group, both of which are optimization-seeking variables; Y_{c,p} is the spatial localization coordinates of the p-th feature point in the c-th viewpoint, and λ_{c,p} is a Boolean value indicating whether the feature p is observed in viewpoint c or not;

Finding the optimal X based on the 3D block leveling method is output as the result of feature point registration.

Further, the spatial localization of marks is specified as, the spatial localization of marks is a fusion of the feature point localization and the registration result, which is obtained by optimizing the following equation:

Σ_{P}^{P}Λ_{P}(X_{P}−DY_{P})²
wherein X is the registered coordinates of the feature point, Y is the spatial localization coordinates of the currently detected feature point; D is the mark positional attitude, an optimization-seeking variable; and the optimal positional attitude D is output as a spatial localization result of the mark.

Further, the applying the Hella code to real-time tumor positioning and tracking based on the above is specifically applied to real-time tumor positioning and tracking after control validation using a radiotherapy pose test;

A position-aware mark point and a metal mark point (metallic mark point) are secured in the same mark point holder, and the position-aware mark point is affixed to the patient's skin where the thermoplastic membrane portion is removed. The meta-mark point serves as an alignment reference during image guidance by the image guidance positioning system IGPS, and the position-aware mark point responds to the distance moved during the pendulum position;

Image guidance with the IGPS corrects for patient posing errors, and the visual localization system tracks the distance moved by the patient during this process, and compares the consistency of the distance moved by the patient calculated by the two.

A distributed multi-camera real-time tumor positioning and tracking system based on visual position-aware mark, the localization and tracking system comprising:

An encoder for encoding a high-density self-recognizing visual mark to obtain a specific Hella code; the encoding: comprising the generation of self-recognizing units, and the splicing of self-recognizing patterns;

A recognition module for detecting and recognizing the Hella code; recognition: including detection of basic features, decoding of self-recognizing units;

A localization and registration module for spatial localization and full-view registration of features of marks marked therewith based on the recognized Hella code; localization and registration: including spatial localization of feature points, spatial localization of marks, full-view registration of feature points, and calibration of the patient's body surface.

Further, the localization and tracking system is applied to tumor positioning and tracking.

The above description is only a preferred embodiment of the present invention, and is not intended to limit the present invention, and any modifications, equivalent substitutions, improvements, etc. made within the spirit and principles of the present invention shall be included in the scope of protection of the present invention and creation.

What is claimed is:

1. A distributed multi-camera real-time tumor positioning and tracking method based on a visual position-aware mark, comprising:
encoding a high-density self-recognition visual mark to obtain a specific Hella code;
detecting and identifying the Hella code;
spatial positioning and full-view registration of a marked feature marked by an identified Hella code;
applying the Hella code to the tumor positioning and tracking based on the above.

2. The method according to claim 1, wherein encoding of the high-density self-recognition visual mark to obtain the specific Hella code comprises generating a self-identification unit and a splicing self-identification unit;
the generating the self-identification unit comprises using an intersection point as a basic feature, with a self-recognition pattern being one basic unit for each 3×3 feature, and using an intersection orientation to compile an identification number;
taking the Boolean value of a central feature orientation as the first bit, starting from the upper left corner feature, filling subsequent bits clockwise to form a 9-bit feature value, and converting the 9-bit feature value into a decimal identification number;
rotating the self-recognition pattern clockwise three times to generate recognition numbers respectively;
by shifting and inverting;
$${\int }_{i}^{90} = \{ \begin{matrix}{f}_{i} & i = 0 \\ \sim {f}_{7 + i} & 3 > i > 0 \\ \sim {f}_{i - 2} & i > 3\end{matrix}$$
wherein fᵢ denotes the i-th bit of the identification number f, f⁹⁰ denotes the identification number of the self-identification unit after clockwise rotation, and ˜ is an inversion operation.

3. The method according to claim 2, wherein the splicing self-identification unit comprises exhausting all the 9-bit feature value, and filling the 9-bit feature value into a unit pool;
then, traversing the unit pool, discriminating and removing unit with repeated rotation and ambiguity based on a shift and inversion operation of the identification number;
and finally, establishing a connection table based on a common of adjacent units regions, and iteratively splicing a self-recognition pattern.

4. The method according to claim 2, wherein the detecting and identifying the Hella code comprises detecting basic feature of a visual mark and decoding of the self-identification unit;
the detecting basic feature of the visual mark comprises feature initial screening, ridge localization, template validation and circular validation, in a fast-to-slow, and layer-by-layer manner that balances real-time and high accuracy;
the feature initial screening is based on anti-color characteristic of the intersection point, checking 8 sampling points and calculating the contrast value to quickly exclude non-intersection pixels:
$$\{ \begin{matrix}{G}_{outer} = {max}_{i} ( {I}_{i} - {I}_{i + 4} ) - {min}_{i} ( {I}_{i} - {I}_{i + 4} ) \\ {G}_{inner} = \vert {I}_{i} - {I}_{i + 4} \vert\end{matrix} i = 1 , … , 8$$
Where Iᵢ is a luminance value of i-th sampling point; locating two ridges of an intersection feature based on a point location;
correcting a sampling center to a center of the intersection point with sub-pixel accuracy based on an intersection position of the ridges to generate the validation frame and extract a local pattern;
first, characterizing the intersection feature by the angle (θ1,θ2) of the ridge, looking up the table in the offline template generated based on multiple sampling, matching with a standard template, and verifying correlation;
then extracting a circular range connected by the intersection feature, computing a sub-pixel luminance center of gravity, and verifying completeness of a circle; and after passing all verifications, outputting an image coordinate of the feature.

5. The method according to claim 4, wherein the decoding of the self-identification unit comprises:
firstly, organizing detected feature points into a longitudinally and horizontally connected graph structure based on a ridge direction, and assigning a relative number (l, m, n);
wherein IϵL isa number of a connected graph, m, and n are temporary coordinates of the detected feature points in a graph structure l;
subsequently, extracting all 3×3 arrays, calculating the identification number based on the intersection orientation, and comparing with a key matrix, calculating a deviation Oₗ, m, n between temporary coordinates and absolute coordinates of the feature;
Finally, recognizing a unique deviation Oₗ for each graph structure based on a majority principle:
O₁=Mode(O_{1,m,n})
wherein Mode denotes the number of plurality; based on a result of deviation voting, the detected feature whose Oₗ,m,n are not equal to Oₗ is excluded; up to this point, decoding of the self-identification unit is completed, and a unique identification number is assigned to each reliably detected feature.

6. The method according to claim 1, wherein the spatial positioning and full-view registration of the marked feature marked by recognized Hella code comprises spatial localization of a feature point, full-view registration of the feature point, and spatial localization of mark;
using at least a four-eye camera set, based on a self-identified feature detected in each view and a camera set calibration data, to complete the spatial localization of the feature point; and
spatially localizing the feature point by full-view angle shooting by means of camera surround or target rotation, and searching for T and X that make the following formula optimal to complete the full-view angle registration of the feature point:
$${\sum }_{C}^{C} {\sum }_{P}^{P} {{λ}_{c , p} ( {Y}_{c , p} - {T}_{C} {X}_{P} )}^{2}$$
wherein cϵC is an observation viewpoint number and pϵP is a feature point number; Tc is a rotational translation matrix of viewpoint c, and X is a registration coordinates of a feature point group, Tc and X are optimization-seeking variables; Y_{c,p} is a spatial localization coordinate of p-th feature point in c-th viewpoint, and λ_{c,p} is a Boolean value indicating whether feature p is observed in viewpoint c or not;
founding an optimal X based on a 3D block leveling method and outputting the optimal X as a feature point registration result.

7. The method according to claim 6, wherein the spatial localization of mark is a fusion of a feature point localization and the registration result, obtained by optimizing the following equation:
$${\sum }_{P}^{P} {⋀}_{P} {( {X}_{P} - {DY}_{P} )}^{2}$$
wherein X is the registration coordinate of the feature point, Y is the spatial localization coordinate of a currently detected feature point; D is a mark positional attitude, which is an optimization-seeking variable; and an optimal positional attitude D is output as a spatial localization result of the mark.

8. The method according to claim 6, wherein applying the Hella code to real-time tumor positioning and tracking after performing control verification by using a radiotherapy positioning test to apply the Hella code to real-time tumor positioning and tracking;
fixing a position-aware mark point and a metal mark point in a same mark point holder, and affixing the position-aware mark point to the patient's skin in a portion where a thermoplastic membrane is removed; using the metal mark point as an alignment reference for image guidance by an image-guidance positioning system IGPS, and using the position-aware mark point to react the distance moved during a pendulum posing;
using the IGPS for image guidance to correct a patient's posing error, and using a visual positioning system to track the patient's moving distance in this process and comparing the consistency of the patient's moving distance calculated by the two.

9. A system for implementing the distributed multi-camera real-time tumor positioning and tracking method based on visual position-aware mark according to claim 1, comprising:
an encoder for encoding a high-density self-recognizing visual mark to obtain a specific Hella code;
a recognition module for detecting and recognizing the Hella code;
a localization and registration module for spatial positioning and full-view registration of marked mark features based on the recognized Hella code.

10. The system according to claim 9, wherein the localization and tracking system is applied for tumor positioning and tracking.
